(19) United States
(12) Patent Application Publication
(10) Pub. No.: US 2011/0196285 A1
(43) Pub. Date: Aug. 11, 2011

(54) Hollow Mesoporous Silica Sphere Coated with Gold and Preparation Method Thereof and Use in Cancer Therapy

(76) Inventors: Dong Chen, Beijing (CN); Yangde Zhang, Hunan (CN); Fangqiong Tang, Beijing (CN); Huiyu liu, Beijing (CN); Linlin Li, Beijing (CN); Xianwei Meng, Beijing (CN); Zongjiu Zhang, Hunan (CN)

(21) Appl. No.: 13/123,337

(22) PCT Filed: Sep. 29, 2009
(86) PCT No.: PCT/CN2009/074306

§ 371 Date: Apr. 8, 2011

(30) Foreign Application Priority Data
Oct. 10, 2008 (CN) 200810224011.2

(51) Int. Cl. A61M 37/00; A61K 9/16; A61K 39/395

(52) U.S. Cl. 604/20; 424/490; 424/277.1

(57) ABSTRACT

The present invention relates to the preparation method of a hollow mesoporous silica sphere coated with gold shell and its use in tumor therapy. In the present invention, the hollow mesoporous silica sphere is made as core and its surface is uniformly coated with the gold shell. The antitumor medicine is loaded in the hollow mesoporous silica sphere and the tumor specific targeting agent is coupled with the surface of the gold shell. The particle size of the hollow mesoporous silica sphere and the thickness of the gold shell are controllable. Based on the Mie Scattering Theory, the hollow mesoporous silica sphere coated with gold shell can adjust its absorption in near-infrared area and convert the light energy of infrared laser into peripheral heat which can kill the malignant tumor cells. The hollow mesoporous silica sphere can be used as a carrier for sustained/controlled release of therapeutic medicine, and the tumor specific targeting agent coupled with the surface of the gold shell can make the medicine have the function of targeting.

### Field of the Invention

The present invention pertains to the technical field of nanomaterials and particularly relates to a composite material with high targeting effect and sustained/controlled release, its preparation method and use in tumor therapy and antitumor medicine.

### BACKGROUND OF THE INVENTION

Malignant tumor is one of the key fatal diseases of human. Following the elevation of industrial level and the deterioration of environment, the number of patients with malignant tumor is on the rise in the world. In the recent more than twenty years, the government of every country in the world has kept increasing investment in the research of malignant tumor and the total medical expenditure for cancer patients has caused huge drain of economic resources which is estimated by experts as RMB14 billion a year. Nevertheless, the curative effect on malignant tumor is still not desirable. Conquering cancers has become the common wishes of all governments and people in the world.

In the recent two decades, thermotherapy has become a regular technique for treatment of tumor. As it doesn't induce the reduction of erythrocytes, leucocytes and hematoblasts, doesn't impair hepatic and renal functions, or doesn't have any serious adverse effect on human body while raising the effective rate of treatment and improving the living quality of patients, it is called by WHO as “Green Therapy”.

Recently, American Halas, J. West et al (D. P. O′Neal, L. R. Hirsch, N. J. Halas, J. D. Payne, J. L.West. Cancer Lett. 2004, 209, 171) adopted silicon dioxide nanoparticle coated with gold nanoshell to absorb near-infrared and generate heat to kill tumor cells. Good results were achieved in the in vitro breast cancer cell experiments and animal experiments (mice). The absorption cross section of these nanoparticles is larger by six magnitudes than that of conventional photosensitizer indocyanine green (ICG). Moreover, unlike conventional photosensitizers, it is seldom photofading and has good biocompatibility.

Overgaard (Overgaard J. Radiobiology for radiation oncologists [M]. London: Earnold, 1993:173˜184) pointed out that the treatment of malignant tumor by thermotherapy alone is prone of relapse. In clinical practice, people begin to shift their attention to the comprehensive treatment by thermochemotherapy. Biological research indicates that thermotherapy may result in fatal destruction of mammal cells and animal and human tumors; and may also improve the curative effect of some chemotherapeutic drugs. The synergy of thermotherapy and chemotherapeutic drugs has attracted wide attention, more and more drugs are found synergistic with thermotherapy, and thermochemotherapy is becoming a noteworthy effective treatment means.

Although preliminary progress has been made in the exploration to the mechanism of thermochemotherapy, no nano material that integrates photo-thermal conversion thermotherapy, loading and sustained release of chemotherapeutic drugs, in vivo imaging and targeted therapy has been reported for the treatment of malignant tumor.

### SUMMARY OF THE INVENTION

The first object of the present invention is to provide a composite material, which comprises a hollow mesoporous silica sphere and a gold shell coated on the surface of the hollow mesoporous silica sphere. Based on the Mie Scattering Theory, the hollow mesoporous silica sphere coated with gold shell can adjust its plasma resonance absorption in near-infrared area and convert the photo-energy of near-infrared laser into peripheral heat which can kill malignant tumor cells. The distribution of the particle size of the sphere is narrow and the thickness of the shell is controllable.

The second object of the present invention is to provide an antitumor medicine with high targeting effect and sustained/controlled release.

The third object of the present invention is to provide a preparation method of the composite material which has the advantages of simple and moderate process, no need of special equipment, low cost and short cycle.

The fourth object of the present invention is to provide a preparation method of the antitumor medicine.

The fifth object of the present invention is to provide the use of the composite material in the treatment of cancers in combination with photothermotherapy. The sixth object of the present invention is to provide the use of the composite material in the treatment of cancers as the material may integrate photothermotherapy, the sustained/controlled release of chemotherapeutic drugs and targeting technology.

The objects of the present invention are realized through the following technical solutions: The composite material provided by the present invention comprises a hollow mesoporous silica sphere and a gold shell coated on the surface of the hollow mesoporous silica sphere. The hollow mesoporous silica sphere provided by the present invention is hollow mesoporous silica nano or submicron sphere. The hollow mesoporous silica nano or submicron sphere serves as the core and is mixed and stirred with a colloidal gold solution to obtain the hollow mesoporous silica nano or submicron sphere coated with gold shell having a controllable thickness through reduction. The foregoing hollow mesoporous silica nano or submicron sphere may be accurately controlled by the method for preparing hollow mesoporous silica nano or submicron sphere, and the thickness of the coated gold shell may be adjusted through controlling the ratio between HAuCl₄ and hollow mesoporous silica nano or submicron sphere.

The composite material provided by the present invention is the hollow mesoporous silica sphere uniformly coated with gold shell on the surface.

The hollow mesoporous silica sphere may also have an inner core, which is a movable silica sphere. In the following text, unless otherwise specified, “hollow mesoporous silica sphere” refers to any hollow mesoporous silica sphere, including the hollow mesoporous silica sphere without an inner core and the hollow mesoporous silica sphere with an inner core, while “hollow mesoporous silica sphere with an inner core” only refers to the hollow mesoporous silica sphere with or having an inner core.

The particle size of the hollow mesoporous silica sphere may be within the range of 44˜1000 nm. The specific surface area of hollow mesoporous silica sphere may be 140˜1000 m2/g. The mesoporous aperture may be 3˜50 nm. The particle size of the movable silica sphere may be >0 nm and <600 nm. The thickness of the shell of the movable silica sphere may be 10˜200 nm. The thickness of the gold shell may be 2˜100 nm. The gold shell has a macroporous structure (as the gold shell does not completely cover the hollow mesoporous silica sphere, the uncovered areas form pores), making for the release of the antitumor medicine.

The antitumor medicine provided by the present invention includes an active ingredient of the antitumor medicine and a carrier. The active ingredient of the medicine is loaded in the carrier. The carrier is the composite material provided by the present invention.

A tumor specific targeting agent may be further coupled with the surface of the gold shell of the composite material. The tumor specific targeting agent may be coupled with the surface of the gold shell before or after the composite material is loaded with the antitumor medicine. The tumor specific targeting agent is tumor specific ligand folic acid or tumor specific antibody.

Alternatively, the medicines for treating other human diseases may be loaded to the composite material.

The preparation method of the composite material provided by the present invention includes the following steps:

1) adding a reducer in a 10⁻⁸˜10⁻³mo1/L HAuC1₄ aqueous solution, and stirring to obtain a colloidal gold solution, wherein the concentration of the reducer in the colloidal gold solution is 10⁻⁸˜10⁻³mol/L;

2) adding hollow mesoporous silica spheres into the colloidal gold solution obtained in Step 1) to get gold-adsorbed hollow mesoporous silica sphere, wherein the concentration of the hollow mesoporous silica spheres in the colloidal gold solution is 10⁻¹−10²mg/ml;

3) adding HAuCl4 in a 10⁻⁴˜10⁻¹mol/L K2CO3 solution wherein the concentration of HAuCl4 in the solution is 10⁻⁸˜10⁻³mol/L, adding the gold-adsorbed hollow mesoporous silica sphere obtained in Step 2) to make the concentration of the gold-adsorbed hollow mesoporous silica sphere in the solution be 10⁻²˜10²mg/mL, and then adding a reducer to make the concentration of the reducer in the solution be 10⁻⁸˜10⁻³mol/L, to obtain hollow mesoporous silica spheres coated with gold shell.

The reducer may be at least one of formaldehyde, dimethylamine-borane, sodium borohydride, hydroxylamine hydrochloride, methanol, citric acid, sodium citrate, sodium hypophosphite, hydrazine and tetramethylolphosphonium chloride.

The preparation method of the antitumor medicine provided by the present invention includes: loading the active ingredient into the composite material through immersion method by using a solution of the active ingredient. The immersion method may include: preparing a solution of the active ingredient of the antitumor medicine, dispersing the dry powder of the composite material into the solution of the active ingredient of the antitumor medicine and stirring to obtain medicine-loaded microsphere; and drying, to obtain the hollow mesoporous silica sphere loaded with the active ingredient of the antitumor medicine and uniformly coated with gold shell on the surface, i.e. the antitumor medicine provided by the present invention.

Before or after the active ingredient of the antitumor medicine is loaded, this preparation method may also include coupling tumor specific antibody or tumor specific ligand folic acid with the surface of the gold shell of the composite material through different chemical modification. The method may include:

1) Coupling tumor specific antibody with the surface of the hollow mesoporous silica sphere uniformly coated with gold shell on the surface: adding thioglycollic acid or its derivatives in a 10⁻²˜10² mg/mL. ethanol solution of the composite material to take reaction wherein the concentration of thioglycollic acid or its derivatives in the solution is 10⁻⁷˜10⁻³mol/L; adding N-hydroxysuccinimide (NHS) and 1-ethyl-3-(3-dimethylaminopropyl) carbodiimide hydrochloride (EDC) into the prepared 10⁻²˜10²mg/mL aqueous solution of the composite material containing carboxylate on its surface to make the concentrations of N-hydroxysuccinimide and 1-ethyl-3-(3-dimethylaminopropyl) carbodiimide hydrochloride in the solution be 10⁻⁷˜10⁻³mol/L, respectively, to obtain the activated composite material after reaction; adding the activated composite material and the tumor specific antibody into a phosphate buffer solution (PBS) for reaction, wherein in the phosphate buffer solution, the concentration of the activated composite material is 10⁻²˜10²mg/mL, and the concentration of the tumor specific antibody is 5x10⁻²˜5x10²mg/mL;

2) coupling the tumor specific ligand folic acid with the surface of the hollow mesoporous silica sphere uniformly coated with gold shell on the surface: adding cysteamine or its derivatives in a 10⁻²˜10²mg/mL ethanol solution of the composite material to take reaction wherein the concentration of cysteamine or its derivatives in the solution is 10⁻⁷˜10⁻³mol/L such that amino-activated composite material is obtained;. dissolving 0.01-10g of folic acid in dimethyl sulfoxide (DMSO) solvent, adding 0.09˜9 g of N-hydroxysuccinimide and 0.05˜5 g of N,N′-dicyclohexylcarbodiimide and stirring to activate folic acid; and then, adding 0.01˜1 g of the amino-activated composite material to the activated folic acid solution to take reaction.

The hollow mesoporous silica sphere uniformly coated with gold shell on the surface in the foregoing coupling of tumor specific antibody or tumor specific ligand folic acid is the hollow mesoporous silica sphere uniformly coated with gold shell on the surface and loaded or not loaded with antitumor medicine.

The method for preparing the hollow mesoporous silica sphere with an inner core may refer to the preparation method in Chinese patent application publication No. CN101121519A. Wherein, according to the method as described in CN101121519A, the molar concentration of hydrofluoric acid may be changed from 1x10⁻³˜5x10⁻¹mol/L of CN101121519A to 1x10⁻⁴˜10x10⁻¹mol/L such that the average mesoporous aperture of the hollow silica sphere with an inner core may be changed from 3˜10 nm to 3˜50 nm, and the specific area may be changed from 140˜500 m2/g to 140˜1000 m2/g. If the molar concentration of ammonia is changed from 0.05˜10 mol/L to 0.01˜10²mol/L, the molar concentration of silicate ester is changed from 0.02˜2 mol/L to 0.01˜20 mol/L and the molar concentration of silane coupling agent is changed from 1 x10⁻⁴˜2x10⁻²mol/L to 1x10⁻⁵˜0.2 mol/L, the particle size may be changed from 100˜1000 nm to 44˜1000 nm.

The plasma resonance absorption of the hollow mesoporous silica sphere coated with gold shell provided by the present invention in the near-infrared area can convert the light energy of near-infrared laser into peripheral heat. The hollow mesoporous silica sphere coated with gold shell is injected into the periphery of malignant tumor cells in human body to kill the malignant tumor cells.

The hollow mesoporous silica sphere coated with gold shell provided by the present invention may be used as a carrier for sustained release of antitumor medicine. In the present invention, the active ingredient of antitumor medicine is loaded in the hollow mesoporous silica sphere coated with gold shell, and a tumor specific targeting agent is coupled with the surface of the hollow mesoporous silica sphere coated with gold shell and loaded with the active ingredient of antitumor medicine. The hollow mesoporous silica sphere coated with gold shell, loaded with the active ingredient of antitumor medicine and coupled with tumor specific targeting agent on the surface is injected into human body, and may target malignant tumor cells by applying the targeting technology. With the help of photothermotherapy and the sustained/controlled release of the active ingredient of the antitumor medicine, it may be used to treat malignant tumor cells in human body.

The active ingredient of the antitumor medicine may be various kinds of substances with antitumor activity. For example, it may be at least one of Adriamycin, Taxol, Docetaxel, Vincristine Sulfate, Fluorouracil, Methotrexatum, Novantrone, Cyclic Adenosine Monophosphate, Cyclophosphamide, Peplomycin Sulfate, Nitrocaphane, Solazigune, Aclarubicin Hydrochloride, Carmustine, Temozolomide, Lomustine, Carmofur, Tegafur, Dactinomycin, Mitomycin, Amsacrine, Amifostine, Cisplatin, Alarelin, Aminoglute-thimide and Chlormethine Hydrochloride, or at least one of the derivatives of the foregoing active ingredients, or at least one of the foregoing active ingredients and their derivatives.

The tumor specific targeting agent may include tumor specific ligand folic acid and tumor specific antibody.

The tumors may include lung cancer, breast cancer, melanoma, colon cancer, pancreatic cancer, glioma, hepatic tumor, pulmonary tumor, bone tumour or adrenal tumor and other solid tumors.

In vitro medicine release test: The dry powder of the hollow mesoporous silica sphere coated with gold shell is dispersed into a medicine solution under ultrasound and stirred to obtain medicine-loaded microsphere. After drying, the dry powder of the hollow mesoporous silica sphere loaded with medicine and coated with gold shell is obtained. 10 mg of the dry powder of the medicine-loaded hollow mesoporous silica sphere coated with gold shell prepared by the foregoing method, or the dry powder of the hollow mesoporous silica sphere coated with gold shell of which surface is further coupled with tumor specific targeting agent is added into PBS (pH=7.4) and stirred. The concentration of the active ingredient of the medicine is determined by ultraviolet spectrophotometry. The microsphere loading rate of this composite medicine loading system is 20%˜50% (mass of the active ingredient of the medicine/mass of the medicine-loaded microsphere), wherein the mass of the medicine-loaded micro sphere is the total mass of the active ingredient and the carrier.

In the animal experiment for malignant tumor, the experimental mice were divided into two groups. One group was a treatment group and the other group was a control group without injection of any medicine. After the mice in the treatment group were intravenously injected with the medicine-loaded multifunctional nano preparation provided by the present invention, they were exposed to 808 nm 4 w/cm² laser radiation for 10 min. The exposure frequency was once every three days. The control group didn't adopt any treatment means. One month later, the average tumor size of the experimental mice in the two groups was compared. Through the comparison, it was obtained that the tumor inhibition rate of the medicine-loaded hollow mesoporous silica sphere coated with gold shell with the functions of high targeting effect and sustained/controlled release provided by the present invention (the multifunctional nano preparation), or the hollow mesoporous silica sphere coated with gold shell of which surface is further coupled with tumor specific targeting agent was 40%˜90%. The tumor inhibition rate is the percentage obtained by dividing the difference between the average tumor size of the experimental mice in the treatment group and the average tumor size of the experimental mice in the control group by the average tumor size of the experimental mice in the control group.

The hollow mesoporous silica sphere coated with gold shell and possessing the functions of high targeting effect and sustained/controlled release provided by the present invention has the following characteristics: (1) the hollow mesoporous silica sphere is coated with a gold shell and has a controllable particle size, a mesoporous structure and a large specific surface area. Medicine enters the hollow mesoporous silica sphere through diffusion and adsorption. The medicine loading rate may be controlled through controlling the particle size of the hollow mesoporous silica sphere and the concentration of the medicine; (2) the gold shell has high functionality and biocompatibility and may easily connect tumor specific ligand folic acid and tumor specific antibody, thereby realizing biological targeting function; (3) the plasma formant of the hollow mesoporous silica sphere coated with gold shell may be easily adjusted to near-infrared area and convert the light energy of near-infrared laser into peripheral heat to kill tumor cells; (4) the hollow mesoporous silica sphere coated with gold shell provided by the present invention may be used as a carrier for sustained release of the antitumor medicine, control the release of the antitumor medicine and combine with thermotherapy to kill tumor cells; (5) the tumor specific targeting agent coupled with the surface may target the medicine-loaded hollow mesoporous silica sphere coated with gold shell to the tumor locations and ultimately realize the integration of photothermotherapy with the sustained/controlled release of chemotherapeutic medicine and targeting technology, to treat cancers.

The hollow mesoporous silica sphere coated with gold shell provided by the present invention may also be used as a carrier for sustained release of other therapeutic medicines and possesses a desirable effect of sustained medicine release. The medicine loading rate and release rate may be controlled through controlling the particle size of the hollow mesoporous silica sphere and the concentration of the medicine. The medicine loading rate of the hollow mesoporous silica sphere provided by the present invention may be 20˜50% by mass. The sustained release of the medicine may last several days.

In the present invention, the surface of hollow mesoporous silica sphere is uniformly coated with gold shell and the surface of the gold shell is coupled with tumor specific targeting agent to get a nano preparation with high targeting effect and sustained/controlled release. This nano preparation, not only its plasma formant can be accurately adjusted and convert light energy into heat but also can load medicine and control the slow release of the medicine. The combination between the coupling of tumor specific targeting agent with the surface and the EPR effect (the increase of permeability of tumor vessels to macromolecular substances and the increase of the macromolecular substances retained and accumulated in tumor) makes the enrichment at tumor locations easier and boosts targeting effect. The preparation may be used as a multifunctional nano preparation which integrates thermotherapy, chemotherapy and targeting and has a broad application prospect in the treatment of malignant tumor.

### BRIEF DESCRIPTION OF THE DRAWINGS

FIG. 1 is a TEM photograph of the hollow mesoporous silica submicron sphere coated with gold shell and having an inner core obtained in Example 1 of the present invention.

FIG. 2 is a temperature rise curve of 10 mg of the hollow mesoporous silica submicron sphere coated with gold shell and having an inner core obtained in Example 1 of the present invention, which is exposed to 35w /cm2 laser radiation for 15 min.

FIG. 3 is a diagram showing sustained medicine release of the hollow mesoporous silica submicron sphere coated with gold shell and having an inner core obtained in Example 1 of the present invention to taxol solution.

### DETAILED DESCRIPTION OF THE EMBODIMENTS

### Example 1

(1) In a 10⁻⁸mol/L HAuC1₄ aqueous solution, formaldehyde is added, stirred and dispersed to get a colloidal gold solution, wherein the concentration of formaldehyde in the colloidal gold solution is 10⁻⁸mol/L. In the prepared colloidal gold solution, hollow silica submicron spheres having particle size of 260 nm are added. The spheres have a mesoporous structure. The average mesoporous aperture is 10 nm. The specific surface area of the spheres is 680 m2/g. In the hollow cavity of the silica submicron sphere, there is a movable spherical silica inner core having particle size of 50 nm. The shell of the movable silica submicron sphere is 20 nm thick. The concentration of the hollow silica submicron sphere in the solution is 10⁻¹mg/ml. After reaction, the hollow mesoporous silica spheres adsorbed with gold and having an inner core are obtained. Then, HAuCl4 is added into a 10⁻⁴mol/L K2CO3 solution, wherein the concentration of HAuCl4 in the solution is 10⁻⁸mol/L. The hollow mesoporous silica submicron spheres adsorbed with gold and having an inner core are added to ensure the concentration of the micro sphere in the solution is 0.2 mg/mL. Then, formaldehyde is added to ensure the concentration of formaldehyde in the solution is 10⁻⁶mol/L and get the hollow mesoporous silica submicron sphere coated with gold shell and having an inner core. The particle size of the sphere is 300 nm. The gold shell has a macroporous structure.

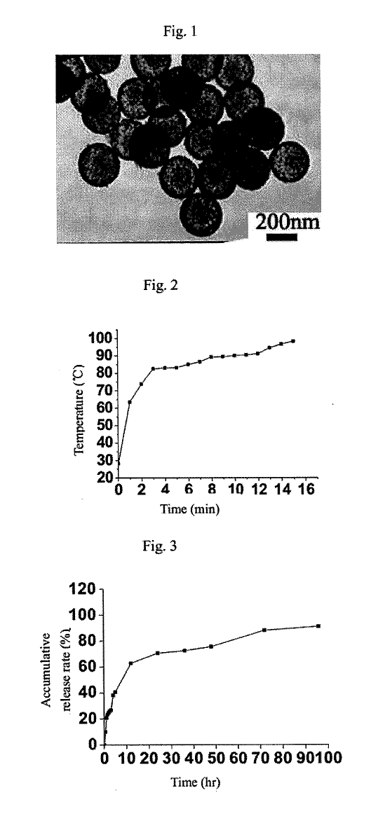

The TEM photograph is shown in FIG. 1. FIG. 2 shows the temperature rise curve of 10 mg of the hollow mesoporous silica submicron sphere coated with gold shell and having an inner core, which is exposed to 35 w/cm2 laser radiation for 15 min. (2) 20 mg/ml docetaxel ethanol solution is prepared. 0.2 g of the dry powder of the hollow mesoporous silica submicron spheres coated with gold shell and having an inner core are dispersed in this docetaxel solution. After stirring, medicine-loaded micro spheres are obtained. After drying, the dry powder of the medicine-loaded hollow mesoporous silica submicron spheres coated with gold shell and having an inner core is obtained. In vitro medicine release test: 10 mg of the multifunctional nano preparation prepared above is put into a dialysis bag. PBS (pH=7.4) is added and stirred. The result is shown in FIG. 3. In a neutral environment, the medicine release rate may reach 91% within 100 h. The medicine loading rate of this multifunctional nano preparation is 50% (medicine mass/mass of medicine-loaded microsphere).

### Example 2

(1) In a 10⁻³mol/L HAuC1₄ aqueous solution, dimethylamine-borane is added, stirred and dispersed to get a colloidal gold solution, wherein the concentration of dimethylamine-borane in the colloidal gold solution is 10⁻³mol/L. In the prepared colloidal gold solution, hollow silica submicron spheres having particle size of 40nm are added. The spheres have a mesoporous structure. The average mesoporous aperture is 7 nm. The specific surface area of the spheres is 520 m2/g. The shell of the hollow silica submicron spheres is 10 nm thick. In the hollow cavity of the silica submicron sphere, there isn't a movable spherical silica inner core. The concentration of the hollow silica submicron spheres in the solutionsuspension is 10²mg/ml. After reaction, the hollow mesoporous silica submicron spheres adsorbed with gold are obtained. Then, HAuCl4 is added into a 0.1 mol/L K2CO3 solution. The concentration of HAuCl4 in the solution is 10⁻³mol/L. The hollow mesoporous silica submicron spheres adsorbed with gold are added to ensure the concentration of the microsphere in the solution suspension is 100 mg/mL. Then, sodium borohydride is added to ensure the concentration of sodium borohydride in the solution is 10⁻³mol/L and get the hollow mesoporous silica submicron spheres coated with gold shell and having an inner core. The particle size of the spheres is 44 nm. The gold shell has a macroporous structure.

(2) The method for evaluating medicine release performance is the same as the method in Example 1. A 2.5 mg/ml cisplatin physiological saline solution is used to replace the docetaxel ethanol solution in Step (2) of Example 1. The result indicates that the medicine release rate may reach about 80% within 140 h. The cisplatin loading rate of the hollow mesoporous silica submicron sphere coated with gold shell is 20%.

### Example 3

(1) In a 2x10⁻⁵mol/L HAuC1₄ aqueous solution, methanol is added, stirred and dispersed to get a colloidal gold solution, wherein the concentration of methanol in the colloidal gold solution is 5x10⁻⁵mol/L. In the prepared colloidal gold solution, hollow silica submicron spheres having particle size of 800nm are added. The spheres have a mesoporous structure. The average mesoporous aperture is 3 nm. The specific surface area of the spheres is 140 m2/g. In the hollow cavity of the silica submicron sphere, there is a movable spherical silica inner core having aprticle size of 600 nm. The shell of the movable silica submicron spheres is 50 nm thick. The concentration of the hollow silica submicron spheres in the is 100 mg/mL. After reaction, the hollow mesoporous silica spheres adsorbed with gold and having an inner core are obtained. Then, HAuCl₄ is added into a 6x10⁻⁷mol/L K2CO3 solution. The concentration of HAuCl4 in the solution is 10⁻⁸mol/L. The hollow mesoporous silica submicron spheres adsorbed with gold and having an inner core are added to ensure the concentration of the microsphere in the suspension is 10⁻²mg/mL. Then, sodium hypophosphite is added to ensure the concentration of sodium hypophosphite in the solution is 6x10⁻⁷mol/L and get the hollow mesoporous silica submicron spheres coated with gold shell and having an inner core. The particle size of the spheres is 1000 nm. The gold shell has a macroporous structure.

(2) The method for evaluating medicine release performance is the same as the method in Example 1. A 15 mg/ml Cephradine aqueous solution is used to replace the docetaxel ethanol solution in Step (2) of Example 1. The result indicates that the medicine release rate may reach about 80% within 200 h. The Cephradine loading rate of the hollow mesoporous silica submicron sphere coated with gold shell is 40%.

### Example 4

(1) In a 4x10⁻⁶mol/L HAuCl4 aqueous solution, hydrazine is added, stirred and dispersed to get a colloidal gold solution, wherein the concentration of hydrazine in the colloidal gold solution is 6x10⁻⁵mol/L. In the prepared colloidal gold solution, hollow silica submicron spheres having particle size of 510 nm are added. The spheres have a mesoporous structure. The average mesoporous aperture is 50 nm. The specific surface area of the spheres is 1000 m2/g. The shell of the silica submicron spheres is 200 nm thick. In the hollow cavity of the silica submicron spheres, there is a movable spherical silica inner core having particle size of 20 nm. The concentration of the hollow silica submicron spheres in the solution is 20 mg/mL. After reaction, the hollow mesoporous silica spheres adsorbed with gold and having an inner core are obtained. Then, HAuCl4 is added into a 1 mol/L K2CO3 solution. The concentration of HAuCl4 in the solution is 10⁻⁷mol/L. The hollow mesoporous silica submicron spheres adsorbed with gold and having an inner core are added to ensure the concentration of the microspheres in the solution is 0.1 mg/mL. Then, sodium citrate is added to ensure the concentration of sodium citrate in the solution is 10⁻⁷mol/L and get the hollow mesoporous silica submicron spheres coated with gold shell and having an inner core. The particle size of the sphere is 600 nm. The gold shell has a macroporous structure.

(2) The method for evaluating medicine release performance is the same as the method in Example 1. A 5 mg/ml adriamycin aqueous solution is used to replace the docetaxel ethanol solution in Step (2) of Example 1. The result indicates that the medicine release rate may reach about 80% within 78 h. The cisplatin loading rate of the hollow mesoporous silica submicron spheres coated with gold shell and having an inner core is 45%.

### Example 5

(1) In a 3x10⁻⁴mol/L HAuCl4 aqueous solution, tetrahydroxymethylphosphonium chloride is added, stirred and dispersed to get a colloidal gold solution, wherein the concentration of tetrahydroxymethylphosphonium chloride in the colloidal gold solution is 6x10⁻⁴ mol/L. In the prepared colloidal gold solution, hollow silica submicron spheres having particle size of 200 nm are added. The spheres have a mesoporous structure. The average mesoporous aperture is 5 nm. The specific surface area of the spheres is 360 m2/g. In the hollow cavity of the silica submicron spheres, there is a movable spherical silica inner core having particle size of 60 nm. The shell of the movable silica submicron spheres is 20 nm thick. The concentration of the hollow silica submicron spheres in the solution is 80 mg/ml. After reaction, the hollow mesoporous silica spheres adsorbed with gold and having an inner core are obtained. Then, HAuCl4 is added into a 0.1 mol/L K2CO3 solution. The concentration of HAuCl4 in the solution is 6x10⁻⁶mol/L. The hollow mesoporous silica submicron spheres adsorbed with gold and having an inner core are added to ensure the concentration of the microspheres in the solution is 10 mg/mL. Then, sodium citrate is added to ensure the concentration of sodium citrate in the solution is 6x10⁻⁶mol/L and get the hollow mesoporous silica submicron spheres coated with gold shell and having an inner core. The particle size of the spheres is 300 nm. The gold shell has a macroporous structure.

(2) The method for evaluating medicine release performance is the same as the method in Example 1. A 2.5 mg/ml cisplatin derivative physiological saline solution is used to replace the docetaxel ethanol solution in Step (2) of Example 1. The result indicates that the medicine release rate may reach about 80% within 150 h. The cisplatin loading rate of the hollow mesoporous silica submicron spheres coated with gold shell and having an inner core is 30%.

### Example 6

(1) In a 7x10⁻⁶mol/L HAuCl4 aqueous solution, sodium borohydride is added, stirred and dispersed to get a colloidal gold solution, wherein the concentration of sodium borohydride chloride in the colloidal gold solution is 6x10⁻⁵mol/L. In the prepared colloidal gold solution, hollow silica submicron spheres having particle size of 420 nm are added. The spheres have a mesoporous structure. The average mesoporous aperture is 6 nm. The specific surface area of the spheres is 400 m2/g. In the hollow cavity of the silica submicron spheres, there isn't a movable spherical silica inner core. The shell of the hollow silica submicron spheres is 200 nm thick. The concentration of the hollow silica submicron spheres in the suspension is 25 mg/ml. After reaction, the hollow mesoporous silica spheres adsorbed with gold and having an inner core are obtained. Then, HAuCl4 is added into an 8x10⁻³mol/L K2CO3 solution. The concentration of HAuCl4 in the solution is 4x10⁻⁷mol/L. The hollow mesoporous silica submicron spheres adsorbed with gold are added to ensure the concentration of the microspheres in the solution is 25 mg/mL. Then, hydrazine is added to ensure the concentration of hydrazine in the solution is 4x10⁻⁷mol/L and get the hollow mesoporous silica submicron spheres coated with gold shell. The particle size of the spheres is 600 nm. The gold shell has a macroporous structure.

(2) The method for evaluating medicine release performance is the same as the method in

Example 1. A 15 mg/ml aqueous solution of the mixture of cisplatin and cisplatin derivatives is used to replace the docetaxel ethanol solution in Step (2) of Example 1. The result indicates that the medicine release rate may reach about 80% within 190 h. The loading rate of the hollow mesoporous silica submicron sphere coated with gold shell to the mixture of cisplatin and cisplatin derivatives is 25%.

### Example 7

The docetaxel-loaded hollow mesoporous silica submicron spheres coated with gold shell, having an inner core obtained in Example 1 are coupled with anti-her2 antibody and used to treat breast cancer beared BALB/c nude mouse model.

1) Docetaxel-loaded hollow mesoporous silica submicron spheres coated with gold shell, having an inner core are coupled with anti-her2 antibody: In a 10⁻²mg/mL ethanol suspension of the docetaxel-loaded hollow mesoporous silica submicron spherse coated with gold shell and having an inner core, thioglycollic acid is added. The concentration of thioglycollic acid in the solution is 10⁻⁷mol/L. After 30 min′s reaction, NHS and EDC are added into the above-prepared 10⁻²mg/mL aqueous suspension of the hollow mesoporous silica spheres coated with gold shell, having an inner core and having carboxylate on the surface to ensure the concentrations of NHS and EDC are both 10⁻⁷mol/L. After 30 min′s reaction, the activated hollow mesoporous silica particles uniformly coated with gold shell on the surface and having an inner core are obtained. anti-her2 antibody is added into the 10⁻²˜10²mg/ml PBS of the obtained activated hollow mesoporous silica particles uniformly coated with gold shell on the surface and having an inner core. The ultimate concentration of anti-her2 antibody is 5x 10⁻² mg/mL. After 2 h′s reaction, a multifunctional nano preparation coupled with anti-her2 antibody, which has the functions of sustained/controlled release and high targeting effect, is obtained.

2) Animal experiment

SK-BR-3 cells are injected to the experimental mice.

The experimental mice were divided into two groups. One group was a treatment group and the other group was a control group without injection of any medicine. After the mice in the treatment group were intravenously injected with 0.5 mg/kg of the medicine-loaded multifunctional nano preparation, they were exposed to 808 nm 4 w/cm2 laser radiation for 10 min. The exposure frequency was once every three days.

The control group didn't adopt any treatment means.

One month later, the average tumor size of the experimental mice in the two groups was compared, indicating that the tumor inhibiton rate of the multifunctional nano preparation was 90%.

### Example 8

The cisplatin-loaded hollow mesoporous silica submicron spheres coated with gold shell obtained in Example 2 are coupled with anti-CD146 antibody AA98 then used to treat lung cancer beared BALB/C mouse model.

1) Cisplatin-loaded hollow mesoporous silica submicron spheres coated with gold shell are coupled with antibody AA98: In a 10²mg/mL aqueous solution of the cisplatin-loaded hollow mesoporous silica submicron sphere coated with gold shell, mercaptopropionic acid is added. The concentration of mercaptopropionic acid in the solution is 10⁻³mol/L. After 30 min′s reaction, NHS and EDC are added into the above-prepared 10⁻²mg/mL aqueous solution of the hollow mesoporous silica sphere coated with gold shell and containing carboxylate on the surface to ensure the concentrations of NHS and EDC are both 10⁻³mol/L. After 30 min′s reaction, the activated hollow mesoporous silica particles uniformly coated with gold shell on the surface are obtained. antibody AA98 is added into the 10²mg/ml PBS of the obtained activated hollow mesoporous silica particles uniformly coated with gold shell on the surface. The ultimate concentration of antibody AA98 is 5x10²mg/mL. After 2h′s reaction, a multifunctional nano preparation coupled with antibody AA98, which has the functions of sustained/controlled release and high targeting effect is obtained.

2) Animal experiment

Lewis lung cancer cells are injected to the armpits of the experimental mice.

The experimental mice were divided into two groups. One group was a treatment group and the other group was a control group without injection of any medicine. After the mice in the treatment group were intravenously injected with 0.5 mg/kg of the medicine-loaded multifunctional nano preparation, they were exposed to 808 nm 4 w/cm2 laser radiation for 10 min. The exposure frequency was once every three days.

The control group didn't adopt any treatment means.

One month later, the average tumor area of the experimental mice in the two groups was compared, indicating that the tumor inhibiton rate of the multifunctional nano preparation was 84%.

Example 9

The adriamycin-loaded hollow mesoporous silica submicron spheres coated with gold shell, and having an inner core obtained in Example 4 are coupled with ligand folic acid of folic acid receptor and used to treat oral squamous carcinoma beared BALB/c nude mouse model.

1) Adriamycin-loaded hollow mesoporous silica submicron spheres coated with gold shell and having an inner core are coupled with folic acid: In a 10⁻²mg/mL, ethanol solution of the adriamycin-loaded hollow mesoporous silica submicron spheres coated with gold shell and having an inner core, cysteamine is added and mixed evenly. The concentration of cysteamine in the solution is lemon. After 30 min′s reaction, amino-activated hollow mesoporous silica sphere uniformly coated with gold shell on the surface and having an inner core is obtained. 0.01 g of folic acid is weighed and dissolved in 20 ml of DMSO. Then, 0.09 g of NHS and 0.0 5 g of DCC are added and stirred to take folic acid activation reaction for 12 h. 0.01 g of the amino-activated adriamycin-loaded hollow mesoporous silica submicron sphere coated with gold shell and having an inner core is added to take reaction for 4h. The hollow mesoporous silica submicron sphere coated with gold shell, having an inner core and coupled with folic acid is obtained.

2) Animal experiment

Oral squamous carcinoma cells are injected to the armpits of the experimental mice. The experimental mice were divided into two groups. One group was a treatment group and the other group was a control group without injection of any medicine. After the mice in the treatment group were intravenously injected with 0.5 mg/kg of the medicine-loaded multifunctional nano preparation, they were exposed to 808 nm 4w/cm2 laser radiation for 10 min. The exposure frequency was once every three days.

The control group didn't adopt any treatment means. One month later, the average tumor size of the experimental mice in the two groups was compared, indicating that the tumor inhibiton rate of the multifunctional nano preparation was 40%.

### Example 10

The docetaxel-loaded hollow mesoporous silica submicron spheres coated with gold shell and having an inner core obtained in Example 1 are coupled with ligand folic acid of folic acid receptor and used to treat melanin cancer beared BALB/c nude mouse model.

1) Docetaxel-loaded hollow mesoporous silica submicron spheres coated with gold shell and having an inner core are coupled with folic acid: In a 10²mg/mL ethanol suspension of the docetaxel-loaded hollow mesoporous silica submicron sphere coated with gold shell and having an inner core, SH-(CH2)3-NH2 is added and mixed evenly. The concentration of SH-(CH2)3-NH2 in the solution is 10⁻³mol/L. After 30 min′s reaction at room temperature, the amino-activated hollow mesoporous silica sphere uniformly coated with gold shell on the surface and having an inner core is obtained. It is cleaned with deionized water twice. 10 g of folic acid is weighed and dissolved in 20 mL of DMSO. Then, 9 g of NHS and 5 g of DCC are added and stirred to take folic acid activation reaction for 12 h. 1 g of the amino-activated docetaxel-loaded hollow mesoporous silica submicron sphere coated with gold shell and having an inner core is added to take reaction for 4 h. The hollow mesoporous silica submicron sphere coated with gold shell, having an inner core and coupled with folic acid is obtained.

2) Animal experiment

Melanin cancer cells are injected to the armpits of the experimental mice. The experimental mice were divided into two groups. One group was a treatment group and the other group was a control group without injection of any medicine. After the mice in the treatment group were intravenously injected with 0.5 mg/kg of the medicine-loaded multifunctional nano preparation, they were exposed to 808 nm 4 w/cm2 laser radiation for 10 min. The exposure frequency was once every three days.

The control group didn't adopt any treatment means.

One month later, the average tumor area of the experimental mice in the two groups was compared, indicating that the tumor inhibiton rate of the multifunctional nano preparation was 60%.

### Example 11

The hollow mesoporous silica submicron spheres coated with gold shell, having an inner core and not loaded with any medicine obtained in Example 1 are coupled with anti-her2 antibody and used to treat breast cancer beared BALB/c nude mouse model. 1) Hollow mesoporous silica submicron spheres coated with gold shell and having an inner core are coupled with anti-her2 antibody: In a 10⁻²mg/mL ethanol solution of the hollow mesoporous silica submicron spheres coated with gold shell and having an inner core, thioglycollic acid is added. The concentration of thioglycollic acid in the solution is lemon,. After 30 min′s reaction, NHS and EDC are added into the above-prepared 10⁻²mg/mL aqueous solution of the hollow mesoporous silica spheres coated with gold shell, having an inner core and containing carboxylate on the surface to ensure the concentrations of NHS and EDC are both lemon. After 30 min′s reaction, the activated hollow mesoporous silica particles uniformly coated with gold shell on the surface and having an inner core are obtained. Anti-her2 antibody is added into the 10 mg/ml PBS of the obtained activated hollow mesoporous silica particles uniformly coated with gold shell on the surface and having an inner core. The ultimate concentration of anti-her2 antibody is 5x 10⁻²mg/ml. After 2 h′s reaction, a multifunctional nano preparation coupled with anti-her2 antibody, which has high targeting effect is obtained.

2) Animal experiment

SK-BR-3 cells are injected to the armpits of the experimental mice. The experimental mice were divided into two groups. One group was a treatment group and the other group was a control group without injection of any medicine. After the mice in the treatment group were intravenously injected with 0.3 mg/kg of the medicine-loaded multifunctional nano preparation, they were exposed to 808 nm 4 w/cm2 laser radiation for 10 min. The exposure frequency was once every three days. The control group didn't adopt any treatment means.

One month later, the average tumor area of the experimental mice in the two groups was compared, indicating that the tumor inhibiton rate of the multifunctional nano preparation was 70%.

### Example 12

1) The docetaxel-loaded hollow mesoporous silica submicron spheres coated with gold shell obtained in Example 3 are used to treat lung cancer beared BALB/C mouse model.

2) Animal experiment

Lewis lung cancer cells are injected to the armpits of the experimental mice. The experimental mice were divided into two groups. One group was a treatment group and the other group was a control group without injection of any medicine. After the mice in the treatment group were intravenously injected with 0.5 mg/kg of the medicine-loaded multifunctional nano preparation, they were exposed to 808 nm 4 w/cm2 laser radiation for 10 min. The exposure frequency was once every three days.

The control group didn't adopt any treatment means.

One month later, the average tumor area of the experimental mice in the two groups was compared, indicating that the tumor inhibiton rate of the multifunctional nano preparation was 54%.

1. A composite material, comprising a hollow mesoporous silica sphere and a gold shell coated on the surface of the hollow mesoporous silica sphere.

2. The composite material as in claim 1, wherein the hollow mesoporous silica sphere has an inner core which is a movable silica sphere.

3. The composite material as in claim 1, wherein the particle size of the hollow mesoporous silica sphere is within the range of 44˜1000 nm, the specific surface area of the hollow mesoporous silica sphere is 140˜1000 m2/g, the mesoporous aperture is 3˜50 nm, the thickness of the gold shell is 2˜100 nm and the gold shell has a porous structure.

4. The composite material as in claim 2, wherein the particle size of the movable silica sphere is above 0 nm and not larger than 600 nm, and the shell of the movable silica sphere is 10˜200 nm thick.

5. An antitumor medicine, containing an active ingredient of the antitumor medicine and a carrier, wherein the active ingredient is loaded in the carrier, and the carrier is the composite material as in claim 1.

6. The medicine as in claim 5, wherein the medicine further contains a tumor-specific targeting agent which is coupled with the surface of the gold shell of the composite material.

7. The medicine as in claim 6, wherein the tumor specific targeting agent is tumor specific ligand folic acid or tumor specific antibody.

8. The medicine as in claim 5, wherein the active ingredient is at least one selected from Adriamycin, Taxol, Docetaxel, Vincristine Sulfate, Fluorouracil, Methotrexatum, Novantrone, Cyclic Adenosine Monophosphate, Cyclophosphamide, Peplomycin Sulfate, Nitrocaphane, Solazigune, Aclarubicin Hydrochloride, Carmustine, Temozolomide, Lomustine, Carmofur, Tegafur, Dactinomycin, Mitomycin, Amsacrine, Amifostine, Cisplatin, Alarelin, Aminoglute-thimide and Chlormethine Hydrochloride; or at least one selected from the derivatives of the foregoing active ingredients; or at least one selected from the foregoing active ingredients and their derivatives.

9. A preparation method of the composite material as in claim 1, wherein the method includes the following steps:
1) adding a reducer in a 10⁻⁸˜10⁻³mol/L HAuCl₄ aqueous solution, and stirring to obtain a colloidal gold solution, wherein the concentration of the reducer in the colloidal gold solution is 10⁻⁸˜10⁻³mol/L;
2) adding hollow mesoporous silica spheres into the colloidal gold solution obtained in Step 1) to get gold-adsorbed hollow mesoporous silica sphere, wherein the concentration of the hollow mesoporous silica spheres in the colloidal gold solution is 10⁻¹˜10²mg/ml;
3) adding HAuCl4 in a 10⁻⁴˜10⁻¹ mol/L K2CO3 solution wherein the concentration of HAuCl4 in the solution is 10⁻⁸˜10⁻³mol/L, adding the gold-adsorbed hollow mesoporous silica sphere obtained in Step 2) to make the concentration of the gold-adsorbed hollow mesoporous silica sphere in the solution be 10⁻²˜10²mg/mL, and then adding a reducer to make the concentration of the reducer in the solution be 10⁻⁸˜10⁻³mol/L, to obtain hollow mesoporous silica spheres coated with gold shell.

10. The method as in claim 9, wherein the reducer is at least one of formaldehyde, dimethylamine-borane, sodium borohydride, hydroxylamine hydrochloride, methanol, citric acid, sodium citrate, sodium hypophosphite, hydrazine and tetrahydroxymethylphosphonium chloride.

11. A preparation method of the medicine as in claim 5, wherein the preparation method includes: loading the active ingredient into the composite material through immersion method by using a solution of the active ingredient.

12. The method as in claim 11, wherein before or after loading the active ingredient, the method further includes coupling a tumor specific antibody with the surface of the gold shell of the composite material by:
adding thioglycollic acid or its derivatives in a 10⁻²˜10² mg/mL ethanol solution of the composite material to take reaction wherein the concentration of thioglycollic acid or its derivatives in the solution is 10⁻⁷˜10⁻³mol/L; adding N-hydroxysuccinimide and 1-ethyl-3-(3-dimethylaminopropyl) carbodiimide hydrochloride into the prepared 10⁻²˜10²mg/mL aqueous solution of the composite material containing carboxylate on its surface to make the concentrations of N-hydroxysuccinimide and 1-ethyl-3-(3-dimethylaminopropyl) carbodiimide hydrochloride in the solution be 10⁻⁷˜10⁻³mol/L, respectively, to obtain the activated composite material after reaction; adding the activated composite material and the tumor specific antibody into a phosphate buffer solution for reaction, wherein in the phosphate buffer solution, the concentration of the activated composite material is 10⁻²˜10²mg/ml, and the concentration of the tumor specific antibody is 5x10⁻²˜5x10²mg/mL, or before or after loading the active ingredient, the method further includes coupling tumor specific ligand folic acid with the surface of the gold shell of the composite material by:
adding cysteamine or its derivatives in a 10⁻²˜10²mg/mL ethanol solution of the composite material to take reaction wherein the concentration of cysteamine or its derivatives in the solution is 10⁻⁷˜10⁻³mol/L such that amino-activated composite material is obtained;
dissolving 0.01˜10 g of folic acid in dimethyl sulfoxide solvent, adding 0.09˜9 g of N-hydroxysuccinimide and 0.05˜5 g of N,N′-dicyclohexylcarbodiimide and stirring to activate folic acid; and then, adding 0.01 ˜1 g of the amino-activated composite material to the activated folic acid solution to take reaction.

13. Use of the composite material as in claim 1, wherein the plasma resonance absorption of the composite material in the near-infrared area can convert the light energy of near-infrared laser into peripheral heat, and inject the composite material into the periphery of malignant tumor cells in human body to kill the malignant tumor cells.

14. Use of the composite material as in claim 1, wherein the active ingredient of the antitumor medicine is loaded to the composite material, tumor specific targeting agent is coupled with the surface of the composite material loaded with the active ingredient of the antitumor medicine, and the composite material loaded with the active ingredient of the antitumor medicine and coupled with tumor specific targeting agent on the surface is injected into human body, such that by applying the targeting technology, the composite material loaded with the active ingredient of the antitumor medicine and coupled with tumor specific targeting agent on the surface can target malignant tumor cells, and is used to treat malignant tumor cells in human body with the help of combined photothermotherapy and the sustained/controlled release of the active ingredient of the antitumor medicine.

15. The composite material as in claim 2, wherein the particle size of the hollow mesoporous silica sphere is within the range of 44˜1000 nm, the specific surface area of the hollow mesoporous silica sphere is 140˜1000 m2/g, the mesoporous aperture is 3˜50 nm, the thickness of the gold shell is 2˜100 nm and the gold shell has a porous structure.

16. The medicine as in claim 5, wherein the hollow mesoporous silica sphere has an inner core which is a movable silica sphere.

17. The medicine as in claim 5, wherein the particle size of the hollow mesoporous silica sphere is within the range of 44˜1000 nm, the specific surface area of the hollow mesoporous silica sphere is 140˜1000 m2/g, the mesoporous aperture is 3˜50 nm, the thickness of the gold shell is 2˜100 nm and the gold shell has a porous structure.

18. The medicine as in claim 16, wherein the particle size of the movable silica sphere is above 0 nm and not larger than 600 nm, and the shell of the movable silica sphere is 10˜200 nm thick.

19. The medicine as in claim 16, wherein the particle size of the hollow mesoporous silica sphere is within the range of 44˜1000 nm, the specific surface area of the hollow mesoporous silica sphere is 140˜1000 m2/g, the mesoporous aperture is 3˜50 nm, the thickness of the gold shell is 2˜100 nm and the gold shell has a porous structure.
